(19) United States
(12) Patent Application Publication
(10) Pub. No.: US 2004/0169673 A1
(43) Pub. Date: Sep. 2, 2004

(54) Graphical user interface for computer-assisted surgery

(75) Inventors: Josiane Crampe, Montreal (CA); Franck Maras, Montreal (CA); Francois Poulin, Montreal (CA); Cynthia Reinert, Montreal (CA)

Correspondence Address: OGILVY RENAULT, 1981 MCGILL COLLEGE AVENUE, SUITE 1600, MONTREAL, QC, H3A2Y3, CA

(73) Assignee: ORTHOsoft INC., Montreal (CA)

(21) Appl. No.: 10/792,730

(22) Filed: Mar. 5, 2004

Related U.S. Application Data
(63) Continuation-in-part of application No. 10/222,832, filed on Aug. 19, 2002.

(51) Int. Cl. A61B 5/00; A61B 5/05; G09G 5/00; G06F 17/60

(52) U.S. Cl. 345/700; 600/300; 705/2

(57) ABSTRACT

A system and a method for performing a computer assisted surgery (CAS) uses an expert system driven graphical user interface (GUI) that displays a series of display pages that provide information related to respective steps required to perform the surgery. The system displays virtual images of surgical instruments used during the surgery, overlaid on fluoroscopic images of the implant site to assist the surgical team during instrument calibration, and implant preparation and installation. The GUI presents the surgical team with a succession of options that the surgical team responds to using affirmation and negation actions. These actions are associated with an icon presented in a same region of the display pages, and are annotated with text relevant to an immediately presented option, permitting a substantial portion of the CAS to be effected using a foot-operated input device.

### CROSS-REFERENCE TO RELATED APPLICATIONS

This is a continuation-in-part of U.S. patent application Ser. No. 10/222,832 filed Aug. 19, 2002.

### MICROFICHE APPENDIX

Not Applicable.

### TECHNICAL FIELD

The present invention relates in general to computer-assisted surgery, and, in particular to a graphical user interface, method and system for facilitating an orthopedic surgical procedure with guidance from an expert system.

### BACKGROUND OF THE INVENTION

Orthopedics is a branch of medicine concerned with diseases, injuries, and conditions of the musculoskeletal system. A large number of orthopedic surgeries are performed each day. To be optimally successful and efficient an orthopedic surgery requires, in addition to a professional surgical team, perfect instruments, imaging support for planning and performing the surgery, and precise control of each step of the surgery. These requirements are especially important when performing an orthopedic surgery using implants (for example screws, such as pedicle screws used in spinal surgery) because a misplaced implant may cause serious harm to the patient, and further may fail to achieve its desired function.

Currently, in some types of orthopedic surgery including spinal operations, a screw hole position is assessed by radiographic imaging and curette palpation. It is recommended that holes be palpated with a curette, or by setting an electromyographic or fibroscopic probe, before screw installation. Furthermore, confirmation of screw placement requires intraoperative radiographing. In some types of orthopedic surgery more than one screw is placed into a patient. The variety of types of orthopedic surgery requires different surgical instruments and implants in a plurality of sizes and types. All of this makes the job of a surgical team more complicated. Some techniques for surgical operations employ a computerized surgical assistance system that uses orthogonal X-ray imaging of the part of the patient of interest in order to simplify the tasks of the surgical team. As is known in the art, installation of pedicle screws, hip replacements, knee replacements, and various other orthopedic, orthodontic and neurological procedures can be assisted using computer technology.

An example of a computerized surgical assistance system is described in U.S. Pat. No. 6,450,978 entitled INTERACTIVE COMPUTER-ASSISTED SURGICAL SYSTEM AND METHOD THEREOF, which issued to Brosseau et al. on Sep. 17, 2002. Brosseau et al. describe a computer-assisted surgical system and method in which a computer includes three-dimensional models of anatomical structures and a user interface including a position sensing system to register in real-time the relative positions of the anatomical structures of interest and of a surgical tool. Interactions between the tool and the anatomical structure are displayed on a monitor using the three-dimensional models. Multi-view display, transparency display and use of cutting planes allow the surgeon to visualize the interaction between the tool and the anatomical structures any time during the surgical procedure. The system can also predict the constraint on anatomical structures before surgery.

Many other computer-assisted surgery systems are known and widely used, especially systems that are particularly useful or explicitly adapted for use in orthopedic surgery. An example is U.S. Pat. No. 5,305,203, entitled COMPUTER-AIDED SURGERY APPARATUS, which issued Apr. 19, 1994 to Raab. Raab teaches a computer-aided surgical device for aiding a surgeon in positioning a surgical instrument (power or manual) when performing surgery on unexposed and exposed portions of a patient. A rudimentary graphical user interface provides geometric diagrams to assist a surgeon in guiding a surgical instrument.

While all such systems provide a user interface, they depend on the expertise of the surgeon to guide the surgical process. As is well known, modern surgery is performed by skilled teams that cooperate to accomplish the task as accurately and efficiently as possible. However, current computer-assisted surgery systems lack an expert system core that is adapted to capitalize on the expertise of team members.

Therefore there exists a need for a computer-assisted surgery system with a graphical user interface that can be used by a surgical team to facilitate a surgical procedure using the expert system. The GUI preferably provides an interface that is simple to use, and integrated with the expert system core.

### SUMMARY OF THE INVENTION

It is therefore an object of the invention to provide a computer-assisted surgery system with a graphical user interface (GUI) adapted to guide a surgical team through a surgical procedure. The GUI provides an interface that is simple to use, and integrated with the expert system core.

### BRIEF DESCRIPTION OF THE DRAWINGS

Further features and advantages of the present invention will become apparent from the following detailed description, taken in combination with the appended drawings, in which:

FIG. 1 schematically illustrates a system for performing computer-assisted surgery (CAS) that includes a graphical user interface (GUI) in accordance with the invention;

FIG. 2 is a flow chart illustrating principal steps of a method for guiding the surgical team in performing a CAS procedure via a GUI;

FIG. 3 schematically illustrates an organization of content of a main menu of the GUI used for computer assisted surgery;

FIG. 4 is a schematic view of a main menu display page in a GUI used for computer assisted surgery, in accordance with an embodiment of the invention;

FIG. 5 is a schematic view of a display page in the GUI for guiding a surgical team during the calibration of an instrument;

FIG. 6 is a schematic view of a second instance of the main menu display page shown in FIG. 4;

FIG. 7 is a schematic view of a display page in the GUI for guiding the surgical team through acquisition of images of the patient;

FIG. 8 is a schematic view of a display page in the GUI for guiding a surgical team during the validation of an acquired image;

FIG. 9 is a schematic view of a display page of the GUI for guiding a surgical team during the preparation of an implant site; and

FIG. 10 is a schematic view of a display page of the GUI for guiding the surgical team through installation of an implant.

It should be noted that throughout the appended drawings, like features are identified by like reference numerals.

### DETAILED DESCRIPTION OF THE PREFERRED EMBODIMENT

The invention provides a simplified user interface for guiding a surgical team through a computer-assisted surgery (CAS) procedure driven by an expert system.

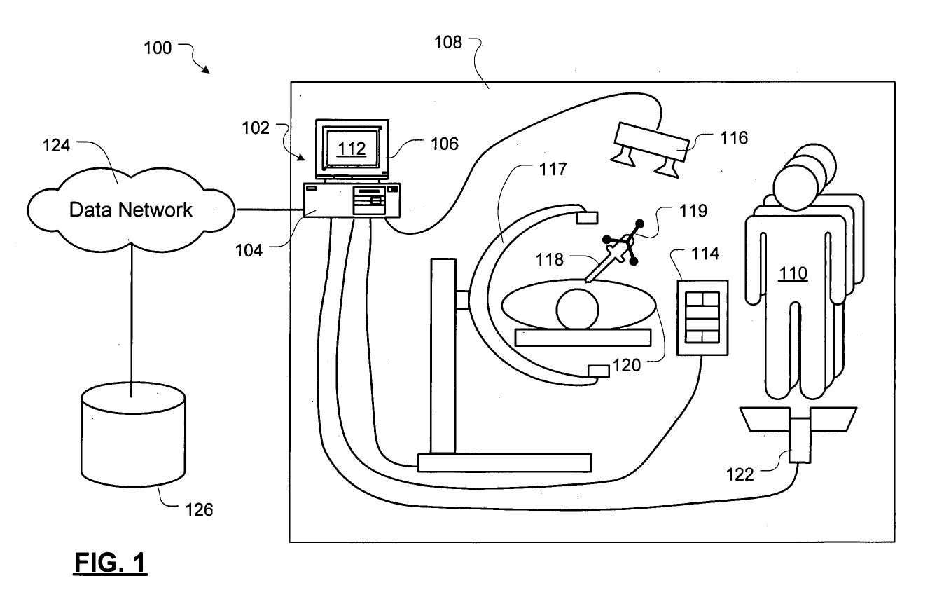

FIG. 1 shows an exemplary embodiment of a system 100 for performing a CAS, in accordance with an embodiment of the invention. The system 100 includes a computer 102 in an operating room 108. The computer 102 has a processor 104 for executing a CAS application and a display monitor 106. The display monitor 106 presents information to a surgical team 110 in a graphical user interface (GUI) 112. In particular, the display monitor 106 is a video display adapted to display images in real-time. The GUI 112 includes a plurality of display pages associated with respective steps required to perform the surgical procedure. Some of the visual display pages display virtual images of selected surgical instruments 118 used during the surgical procedure overlaid on fluoroscopic images of a part of a patient 120 that is subject to the surgical procedure. A manual input device is preferably connected to the computer 102 to permit the surgical team 110 to input commands to the CAS program for advancing through the series of display pages of GUI 112, as each of the respective steps of the surgical procedure is successively completed. The manual input device may be a keypad 114 that is easily sterilized and resistant to fluid contamination. As is known to the persons skilled in the art, the manual input device, like the keypad, can be placed in a sterilized plastic bag. A similarly adapted foot-operated input device 122 may also be connected to the computer 102. The foot-operated device 122 preferably includes two pedals, one associated with an affirmation action, and the other with a negation action. Likewise two keys of the keypad 114 are associated with respective affirmation and negation actions. The surgical team 110 may choose to operate either the keypad 114 or the foot-operated input device 122, as required. The computer 102 may further be connected to a mouse or like user input device.

The computer 102 is connected to a tracking system that includes a binocular video camera 116 for locating and tracking position, orientation and movement of the surgical instrument(s) 118 which are secured to instrument locator(s), as will be explained below in more detail.

An imaging system, such as a fluoroscope 117 (a well known X-ray imaging system)., acquires oriented fluoroscopic images of a part of a patient 120 that is subject to the surgical procedure. As will be understood by those skilled in the art, other imaging systems can also be used such as any one of CT-Scan, MRI, PET, ultrasound and Echography. The images acquired by the fluoroscope 117 are provided to the computer 102, in accordance with the invention. As many oriented fluoroscopic images as are required for the surgical procedure are acquired. The fluoroscopic images are processed by the CAS to provide views of the part of the patient 120 that are aligned with and scaled to the tracking system. Respective fluoroscopic images are then displayed in corresponding image content fields of display pages, and provide a visual guide for the surgical team 110 in the preparation and installation of an implant, as will also be explained below in more detail. The computer 102 may also be connected to a data network which may be a data network such as the Internet 124 or a local area network (LAN) for accessing a remote data source 126 that stores expert systems or applications, externally of the operating room 108. Alternatively, the expert system applications are stored in a memory of the computer 102.

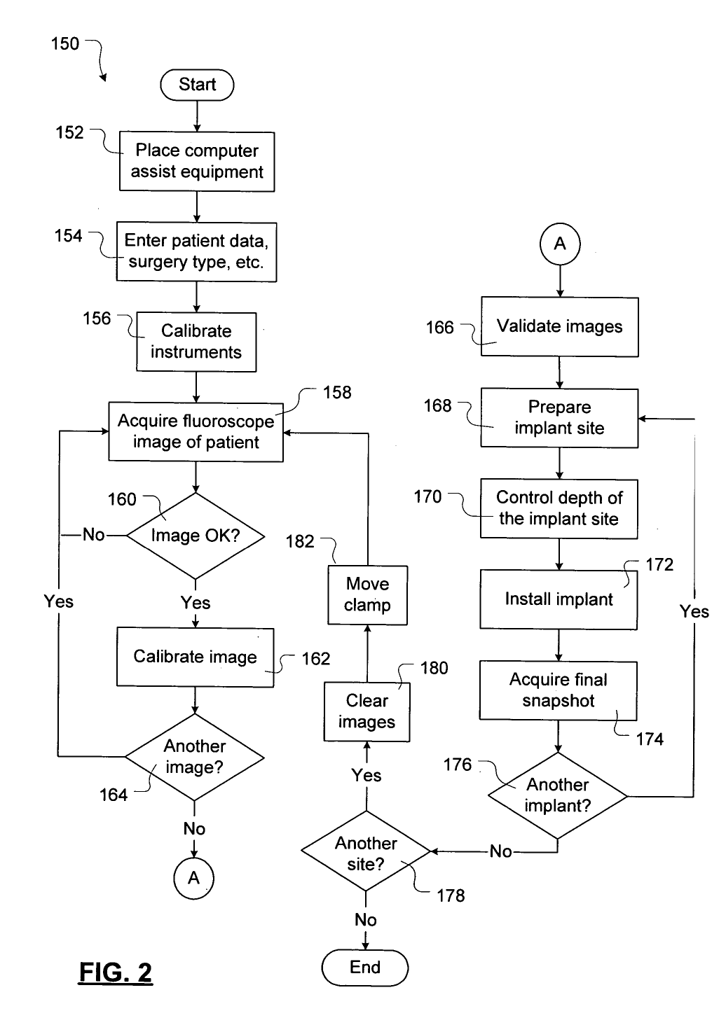

The invention also provides a method for guiding the surgical team 110 in performing a CAS procedure using the GUI 112. A general overview of the method is described with reference to a flow chart 150 shown in FIG. 2. The method provides the surgical team 110 with information related to respective steps required to perform the surgical procedure using the GUI 112. Virtual representations of selected surgical instruments (herein “virtual instruments”) within a field of view of the tracking system are overlaid on the images of the part of the patient 120 that is subject to the surgical procedure. In addition, a series of display pages presented by the GUI 112 guide the surgical team 110 through the CAS procedure, as each of the respective steps is completed. One embodiment of the GUI 112 provides visual and audio information related to instrument calibration, patient imaging, implant site preparation, and implant installation.

The method starts (step 152) by placing the CAS equipment 102 in the operating room 108. Patient data, surgery type, and other information is then entered into the computer 102 (step 154). The surgical instruments 118 are then calibrated (step 156). During the calibration step, the surgical team 110 is prompted to identify a surgical instrument to be calibrated and to connect a three-dimensional instrument locator 119 to the identified instrument 118. In one embodiment, the instrument locator 119 is a light-reflective reference tool. When the identified instrument 118 is secured to the instrument locator 119, the instrument is moved into a field of view of the binocular video camera 116 of the tracking system, so that images of the instrument locator 119 can be used to automatically calibrate the instrument.

The surgical team 110 is (step 158) prompted to effect the acquisition of one or more differently oriented fluoroscopic images of the part of the patient subject to the surgical procedure. The resulting fluoroscopic images are then verified (step 160). If an image is not satisfactory, (i.e. there is unacceptable contrast, the orientation is incorrect, etc.) the expert system returns to step 158 to permit a substitute image to be acquired. Otherwise, the expert system advances to step 162, and the image is calibrated. If more images are required for the surgical procedure (as determined in step 164), the expert system returns to step 158. Otherwise, the expert system advances to step 166. Menu options may be provided to permit the surgical team 110 to transform images, for example by selecting options to rotate, change contrast or brightness, flip or restore the fluoroscopic image.

In step 166, the fluoroscopic images are validated by the surgical team 110 with the guidance of the expert system. The GUI 112 prompts the surgical team to place one of the calibrated instruments on a part of the patient that is subject to the surgical procedure, and to compare the actual location of the surgical instrument 118 with the virtual instrument superimposed on the image of the part of the patient. If the alignment between the calibrated instrument and image of the virtual instrument is not acceptable, the image is deleted and the procedure returns to step 158 where another fluoroscopic image is acquired.

Once the fluoroscopic images required for the procedure have been calibrated and validated, the surgical team 110 is then guided through the preparation of implant sites (step 168). The GUI 112 prompts the surgical team 110 to position a calibrated drill guide or an awl tip connected to a calibrated instrument handle (for example) on the part of the patient where an implant is to be inserted. The site preparation is facilitated by the images which display the fluoroscopic image or images of the part of the patient, and by the virtual instruments. Preferably an axis of the virtual instrument is displayed to indicate an orientation of the drill guide or awl tip. The alignment of the position and orientation of the virtual instrument and the fluoroscopic images of the patient permit the surgical team to monitor site preparation, which generally involves drilling a hole to prepare a bone to receive the implant. Planning the implant site to select a position and axis of orientation may be performed as taught in the above-referenced co-pending, co-assigned patent application. Furthermore, during the preparation of the implant site, the GUI 112 may display a depth gauge used to indicate to the surgical team 110 a distance of travel of a drill bit. The surgical team naturally monitors the depth of the hole throughout the preparation of the implant site 170, in part using the GUI 112 that dynamically updates the visual display to indicate to the surgical team 110 the distance of travel of the instrument as the instrument is used to prepare the implant site.

The GUI 112 then prompts the surgical team 110 to insert the implant (step 172). The GUI 112 displays selections to permit the surgical team 110 to advance to an implant installation step, and then displays, in corresponding image content fields, the fluoroscopic images of the part of the patient that were displayed during implant site preparation. The virtual instrument, and an image of the selected implant positioned and oriented with respect to the other elements in view (herein a “virtual implant”) are overlaid on the displayed fluoroscopic images. The virtual path of the implant is computed by the CAS application by tracking a path of the instrument used to insert the implant into the prepared implant site. After the implant is installed, a “snap shot” (screen image saved in a file on the computer hard drive) is generally acquired (step 174). Snap shots may also be acquired at other times during the procedure using a predefined command, keypad 114 key, mouse selection, etc. Fluoroscopic images may also be taken to document the position of the implant(s). If it is determined that another implant is to be inserted at a site visible in the fluoroscopic images (step 176), the expert system returns to step 168. If required, the surgical team may return to step 158 to acquire fluoroscopic images (not shown on the flow diagram) The GUI 112 will prompt the surgical team to clear the image banks if the clamp has been displaced. Otherwise, it is determined (step 178) whether surgery is required at another site that requires repositioning of the vertebral clamp or other position reference tool. If so the image bank is cleared (step 180), and another location is prepared for surgery (step 182). The procedure then returns to step 158 to acquire images of the next site. Otherwise, the surgical team can quit the CAS application.

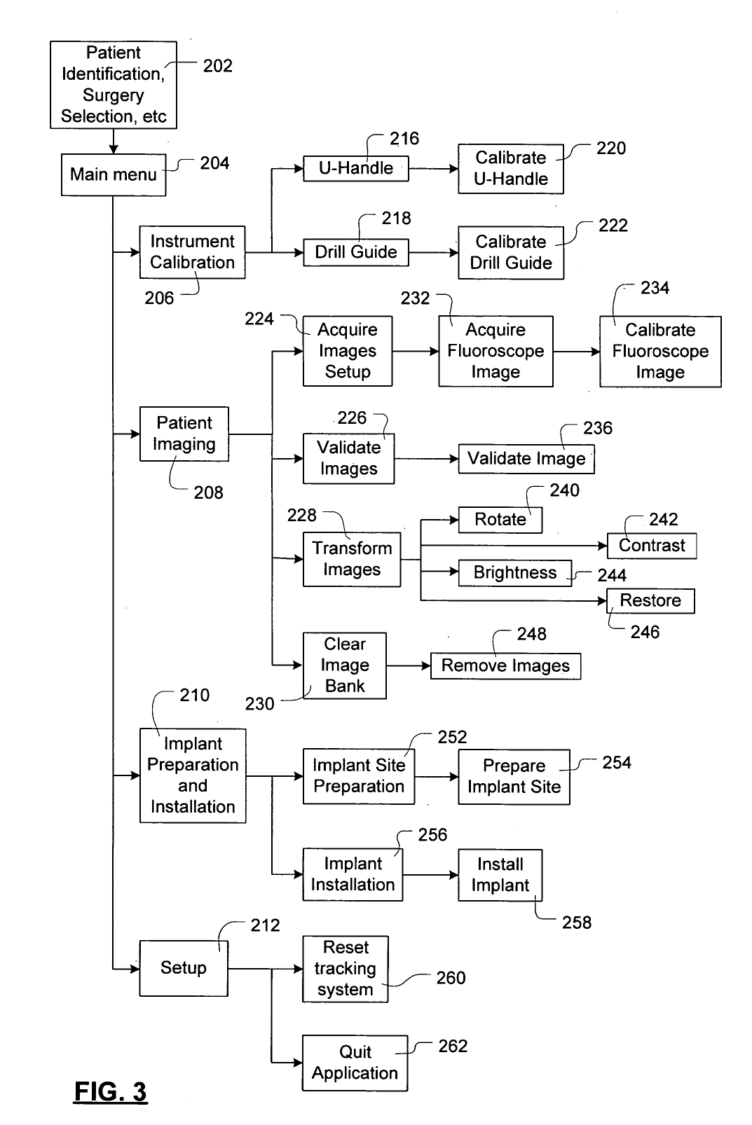

FIG. 3 illustrates content and selections available from a main menu 204 of the GUI 112, in accordance with an embodiment of the present invention. As shown in FIG. 3, the main menu 204 includes a plurality of selections that are organized thematically. The selections provide the surgical team 110 with access to the functionality of the CAS system 100, which is augmented with display pages. Some of the display pages (other than the main menu 204 display page) are also illustrated in FIG. 3.

In the embodiment illustrated in FIG. 3, the GUI 112 provides a main menu 204. It will be noted that the main menu 204 is a display screen with a uniform view that dynamically displays content and selections depending on selections made by the surgical team, and a state of the expert system. As such, selections are adaptively displayed by the main menu 204.

The main menu 204 is accessed after patient information, surgery type, and other information is entered in one or more preliminary pages 202. A patient to be operated on is identified, the type of operation (in the illustrated embodiment, a spinal surgical procedure) is specified and an identification of each member of the surgical team is recorded, along with any other documentary information required. The type of operation is used by the CAS to select an instance of an expert system to drive the GUI 112. Consequently, the type of operation determines a configuration of the remainder of the GUI 112, which may differ from the exemplary structure described with reference to FIG. 3.

The main menu 204 permits the surgical team 110 to access three general categories of functionality, namely: an instrument calibration selection 206 for accessing functionality related to a calibration of selected instruments to be used during the surgical procedure; a patient imaging selection 208 for accessing functionality related to acquiring, processing and validating fluoroscopic images of a part of the patient; an implant preparation and installation selection 210 for accessing functionality related to implant site planning, implant site preparation and implant installation. A setup selection 212 is also provided to permit the surgical team 110 to quit the CAS application, and reset the tracking system. Other selections may also be displayed by the main menu 204 to provide access to other desired functions of the CAS application such as, for example, context-sensitive help. The setup category may be used for selecting an arrangement of the patient, tracking system, fluoroscope, etc. Within each category of functionality, the GUI 112 may include as many display pages as required to enable efficient and intuitive access to the functionality of the CAS application.

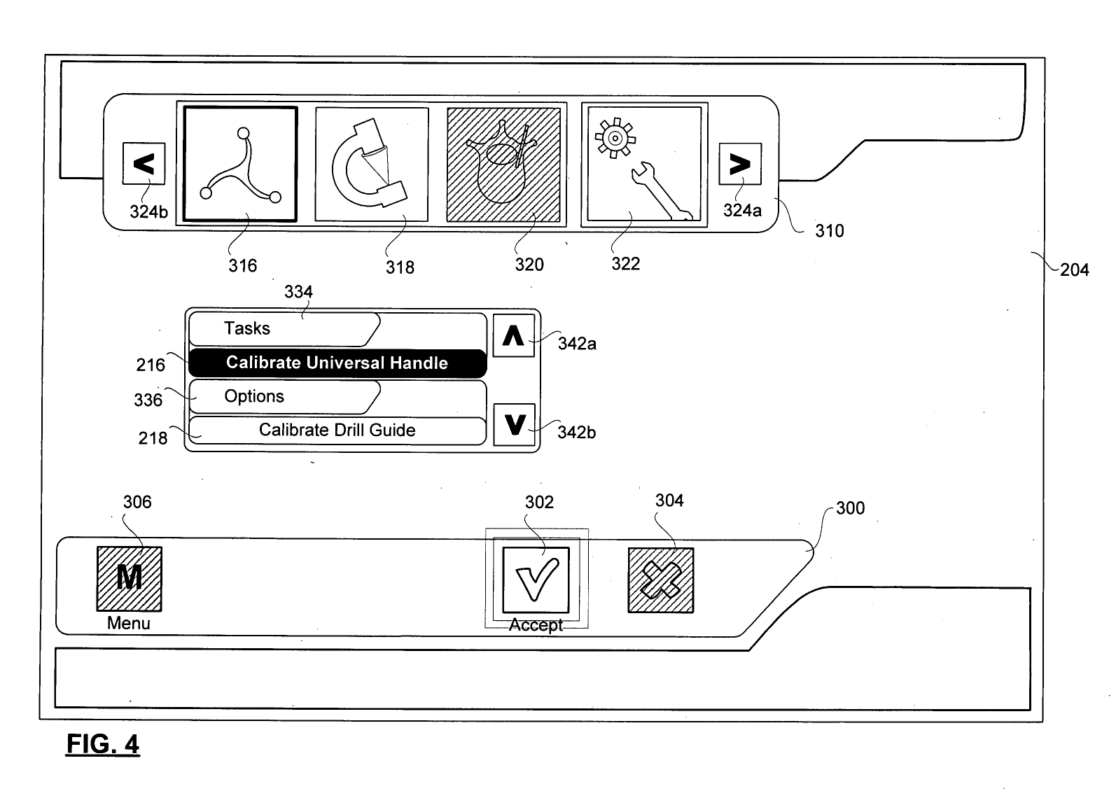

Each of the categories of functionality offered by the selections 206-212 is associated with a corresponding menu bar icon that is used to effect the selections 206-212, as will be described further below with respect to two instances of the main menu 204 illustrated in FIGS. 4,6.

While the expert system guides the surgical team through the steps of the surgical procedure, at any time while the application is running there may be one menu selection suggested by the expert system, one or more allowable but not elicited selection, and one or more displayed but not-selectable option(s). The not-selectable options are displayed as not available by a grayed-out appearance. For example, at a first instance of the main menu 204, the implant preparation and installation selection 210 is not selectable and is grayed-out, as required instruments have not been calibrated, and the required fluoroscopic images have not yet been acquired, calibrated, and verified. Further indications (such as “smileys”, a wizard, assistant, etc.) may be associated with the respective selections 206-210 to indicate which steps have been successfully completed and/or are to be completed. The first step expected by the expert system is the selection and calibration of one or more instruments to be used in the invention.

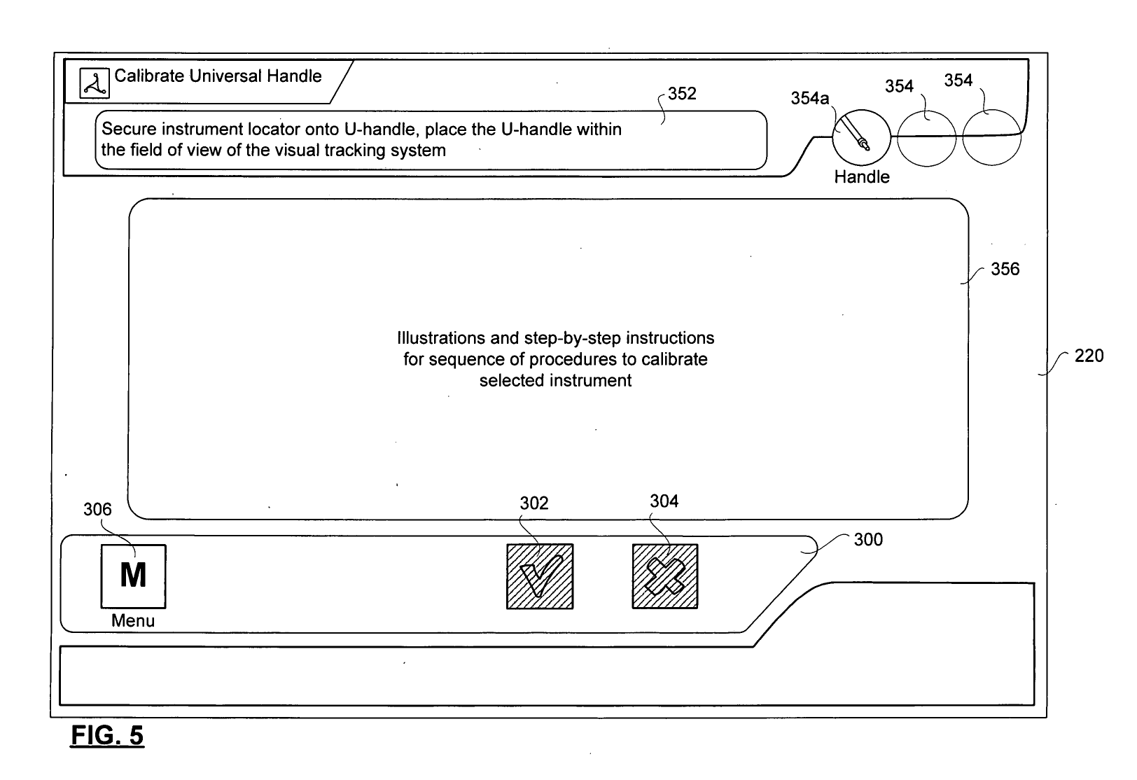

Activating the instrument calibration selection 206 in the main menu 204 updates a means of selections presented to the surgical team. In the illustrated embodiment, these selections includes widgets (selectable icons, buttons, menu options, etc.) for selecting at least one of a list of instruments that may be of use in the surgical procedure for launching respective calibration display pages used to guide the surgical team through the calibration of the associated instrument. Specifically, a U-handle (universal tool handle) selection 216 and a drill guide selection 218, which are useful for orthopedic spinal surgery, are presented. If the U-handle selection 216 is activated, a calibrate U-handle display page 220, an embodiment of which is schematically illustrated in FIG. 5, is displayed. Similarly, if the drill guide selection 218 is activated, a calibrate drill guide display page 222 is displayed. As will be understood by those skilled in the art, more or different instruments may be required for other surgeries such as hip or knee replacements, for example.

Preferably, in accordance with the invention, activation of the U-handle selection 216 or the drill guide selection 218 is effected by an action widget. The action widget is preferably an affirmation action button that is consistently displayed in all display pages in a same position. The affirmation action button is further associated with a respective pedal of the foot-operated device 122, and a key on the keypad 114. The affirmation action button is associated with the acceptance of a currently presented option by the expert system via the GUI 112.

The calibrate U-handle and calibrate drill guide display pages 220,222 preferably include illustrations and instructions for demonstrating how to secure the instrument locator 119 to the instrument, and directions for placing the instrument within a field of view of the tracking system, as will be described further below with reference to FIG. 5. Once an instrument has been calibrated, the expert system again displays instrument calibration selection 206 and suggests a next instrument that is deemed necessary for the surgical procedure. Once all instruments required for the surgical procedure have been calibrated, the expert system may present the main menu 204, with the patient imaging selection 208 highlighted to prompt selection. The surgical team may alternatively choose the instrument calibration selection 206, and calibrate an optional instrument, if desired.

The patient imaging selection 208 of the main menu 204 is used to access functionality of the CAS related to the capturing and processing of images of a surgical site. The patient imaging selection 208 provides an acquire images setup selection 224, a validate images selection 226, a transform images selection 228 and a clear image bank selection 230.

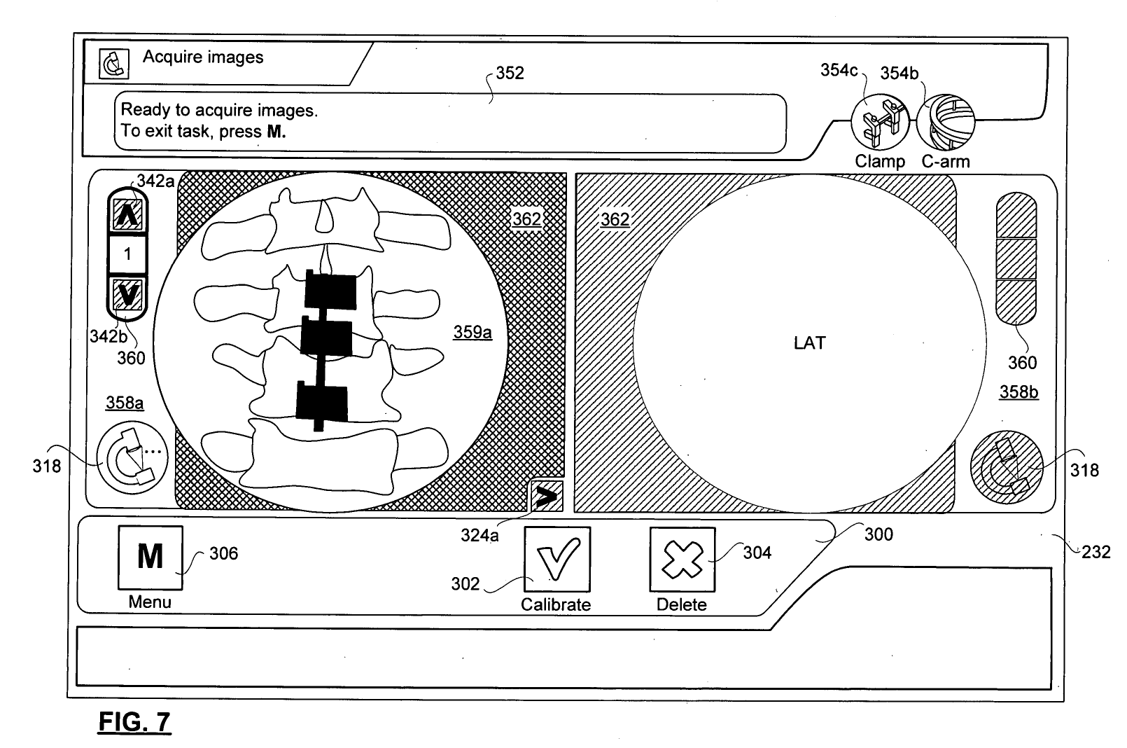

Activation of the acquire images setup selection 224 launches an acquire fluoroscope image display page 232 that guides the surgical team in controlling an imaging system (such as the fluoroscope) of the CAS system to acquire fluoroscopic images. When the imaging system is ready, the surgical team is prompted to acquire a fluoroscopic image using the affirmation action button. The acquired fluoroscopic image is displayed immediately to the surgical team in the image content field of the acquire fluoroscope image display page 232 so that it can be verified, to ensure that adequate resolution of the specific area of interest is achieved. An example of the acquire fluoroscope image display page 232, in accordance with the illustrated embodiment, is shown in FIG. 7. The acquired images are automatically calibrated by activation of an affirmation action button in accordance with an embodiment of the invention. A calibrate fluoroscope image display page 234 showing progress of the calibration procedure is displayed. The calibrated fluoroscopic images of the surgical site is scaled to match the calibrated instruments, and are aligned with the tracking system.

After each fluoroscopic image is calibrated, the patient imaging selection of the main menu 204 is displayed. After a first set of calibrated fluoroscopic images (in this example the fluoroscopic images are a paired anterior-posterior (AP) image and a lateral (LAT) image) is calibrated, the expert system suggests the validate images selection 226, although the surgical team may elect to acquire further images.

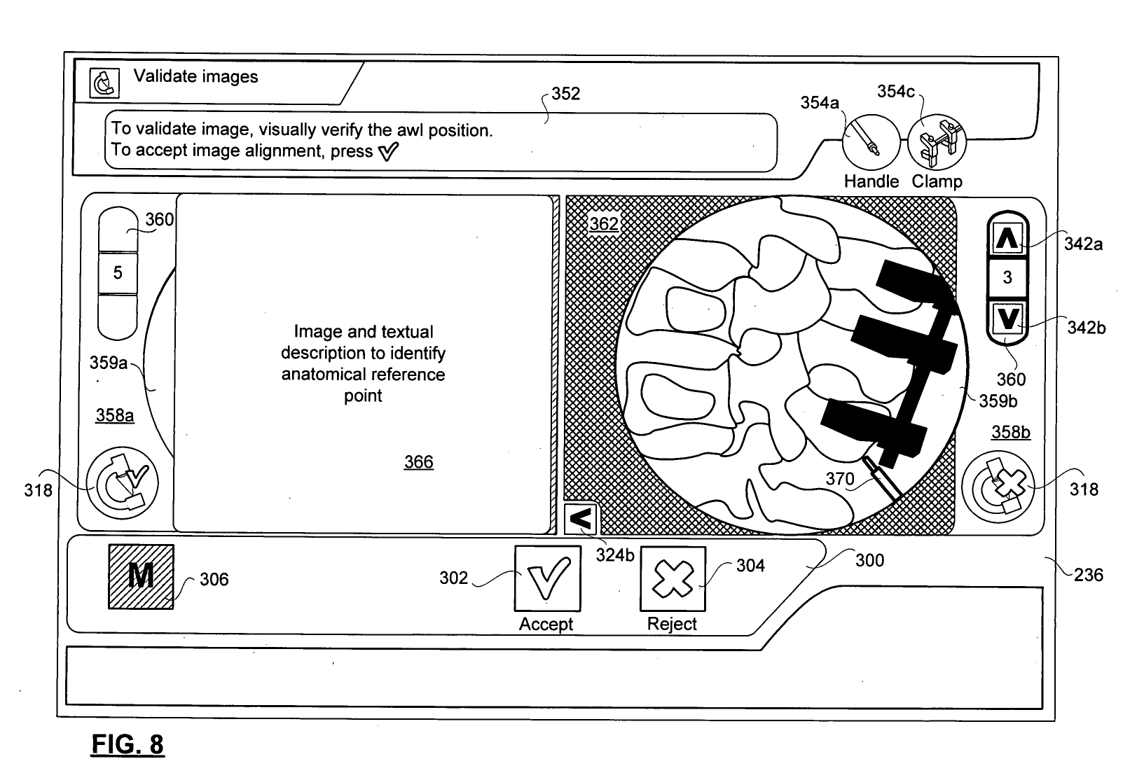

If the validate images selection 226 is selected and the affirmation action button is activated, a validate image display page 236 is launched. The validate image display page 236 displays instructions that enable the surgical team to validate a fluoroscopic image by comparing positions of a calibrated instrument on the part of the person subject to the procedure, with a position of the corresponding virtual instrument on the fluoroscopic image. In accordance with the illustrated embodiment, FIG. 8 shows an exemplary validate image display page 236. The surgical team, following step-by-step instructions can validate the calibrated image to verify that the fluoroscopic image is correctly aligned and scaled with the virtual instrument and the coordinates of the tracking system. After the surgical team has compared a calibrated image with the real points on the surgery site, the surgical team can accept the calibrated image using the affirmation action button, or discard it using a negation action button, that is consistently present in all of the display pages. If the image is accepted, the image is stored in an image bank of a memory of the computer 102. Conversely, if the image is rejected, the image is deleted.

Once the fluoroscopic image is either accepted or rejected, the expert system presents the main menu 204. If there are more images that require validation, the main menu 204 is presented with the patient imaging selection 208 selectable, and the validate images selection 226 suggested. If the required number of fluoroscopic images are not available (i.e. some have been deleted), the acquire images setup selection of the main menu 204 with the patient imaging selection 208 being suggested is displayed. Otherwise, the implant preparation and installation selection 210 is selectable while the implant site preparation selection 252 is suggested.

After a minimum required number of fluoroscopic images have been validated, the surgical team can select the implant preparation and installation selection on the main menu 204. However, it is not until all calibrated images are validated, that the expert system displays the main menu 204 suggesting the implant preparation and installation selection 210. Nonetheless, the surgical team can return to the patient imaging selection 208, and choose a transform images selection 228 which permits the surgical team to modify images by selecting options to rotate an image 240, change a contrast 242 or a brightness 244 of the image, or restore a transformed image 246. Upon completion of the transformation, the surgical team is presented with the same transformation options, and the surgical team can select another fluoroscopic image, and apply one or more other such transformations. When the surgical team has completed the desired transformations, the main menu is selected. The main menu 204 with the implant preparation and installation selection 210 active is presented and the implant site preparation 252 is suggested (assuming a sufficient number of fluoroscopic images have been calibrated and validated).

Whenever an image is calibrated, the surgical team may select from the main menu 204 under the patient imaging selection 208, a clear image bank selection 230 that displays a remove images display page 248. The remove images display page 248 enables the surgical team to delete selected images acquired for the surgical procedure. The GUI 112 preferably displays the images in the image bank to facilitate selection of fluoroscopic images to delete. A remove all images selection on the remove images display page 248 is generally used to restart image acquisition.

When the required number of calibrated and validated fluoroscopic images are present, and the instruments to be used are calibrated, the surgical team is guided to select the implant site preparation selection 252, which is suggested by the main menu 204. The affirmation of the implant site preparation selection 252 launches a prepare implant site display page 254 that guides the surgical team through the preparation of the implant site. At this time, the implant installation selection 256 is not available, and is grayed-out.

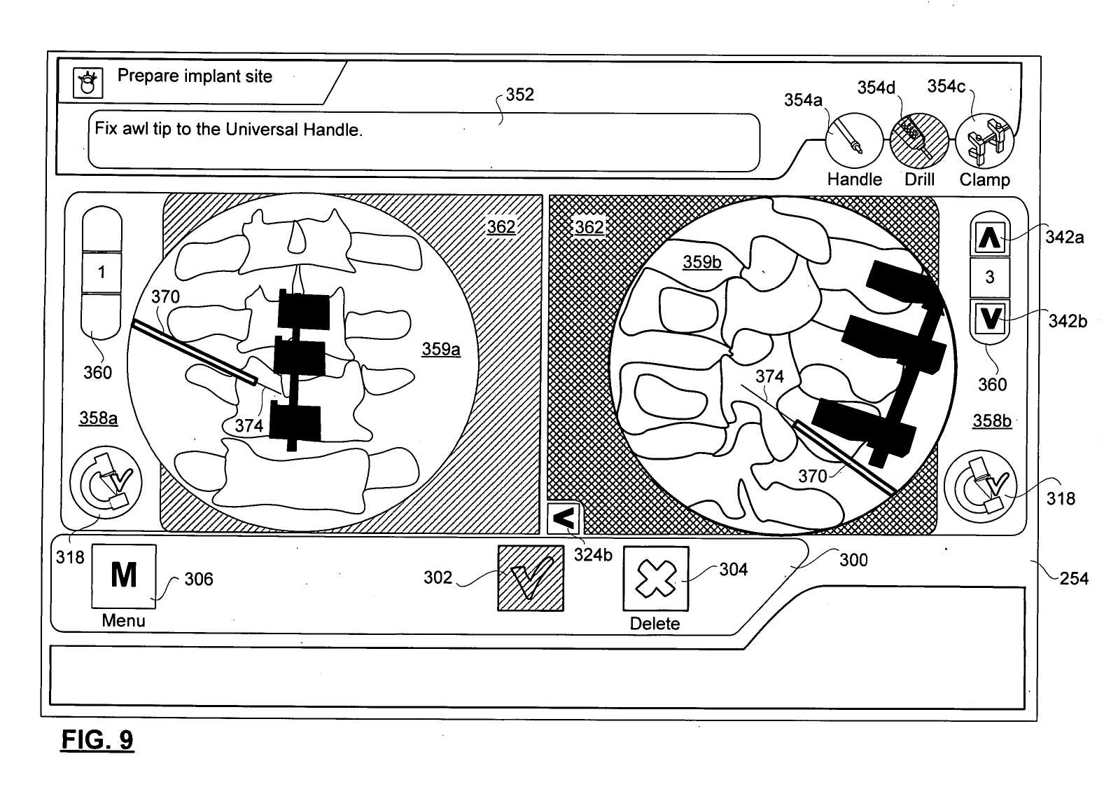

Selection of the implant site preparation selection 252 displays the prepare implant site display page 254. The surgical instrument chosen to prepare the implant site is automatically detected using the instrument locator 119. After the instrument enters the field of view of the tracking system, a status of an icon representing the calibrated instrument is changed. When the surgical instrument enters a field of view of the acquired image, the corresponding virtual instrument is superimposed on the image in both image content fields, as shown in one embodiment illustrated in FIG. 9. When the site is prepared, the surgical team selects the main menu 204 and is returned to the implant preparation and installation selection, in which the suggested action is implant installation 256.

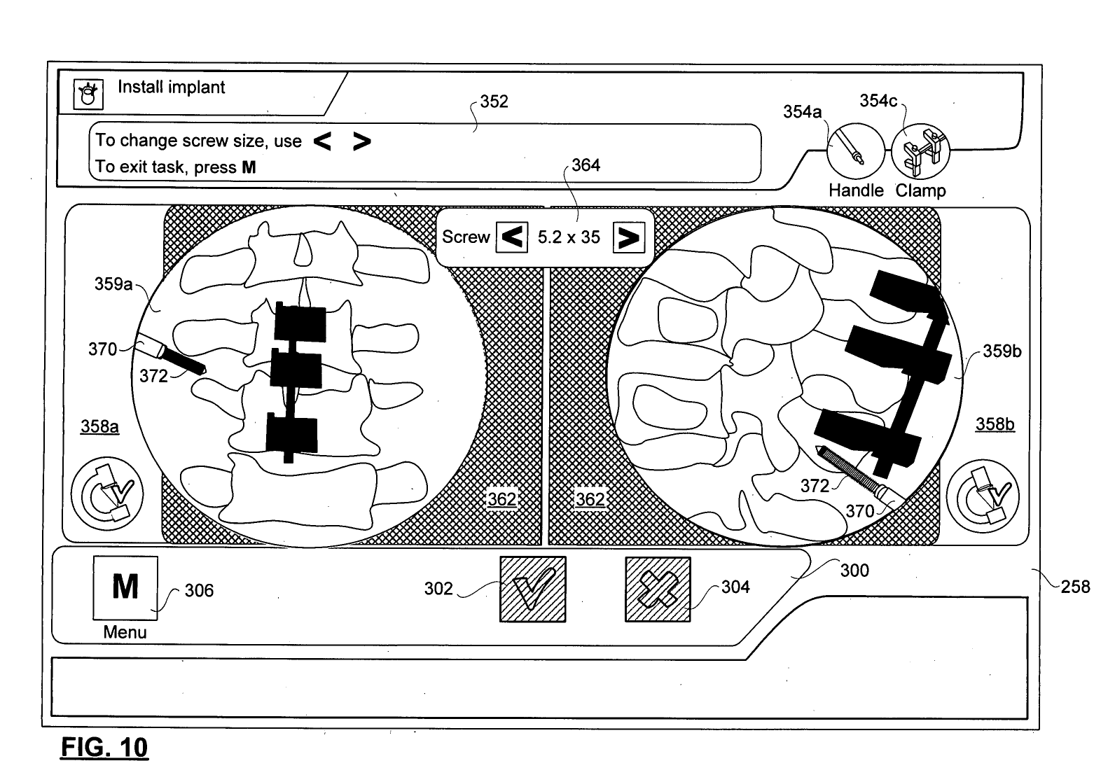

In one embodiment of the invention, a depth gauge may be used to guide the drilling of an implant site. After an implant site is prepared to receive an implant, the surgical team may prepare another implant site or install the implant. When ready, the surgical team is presented with an install implant display page 258, after affirming the implant installation selection 256. The install implant display page 258, an exemplary embodiment of which is illustrated in FIG. 10, guides the surgical team through a process of inserting the implant. A select screw (or implant) size (or size and type) widget is provided to permit the surgical team to select one of a catalog of implants that is to be inserted at the implant site. The virtual instrument and a selected virtual implant are superimposed in real-time over the fluoroscopic images of the part of the patient 120, permitting a visual representation of the actual path of the installed implant and the instrument.

After the implant is installed, surgical records may be completed by acquiring images of the implant using the fluoroscope, for example, or a snap shot of the image presented on the display monitor 106.

When the surgical team selects the setup selection 212 from the main menu 204, a reset tracking system selection 260, and a quit application selection 262 are presented. If the tracking system fails for one reason or another, the surgical team selects the reset tracking system selection 260, which provides a set of options and display pages for troubleshooting and remediation of the tracking system. When the surgical team has completed its procedure the quit application selection 262 is used to exit the program.

Display Page Format

Having described an organizational structure of the GUI 112 shown in FIG. 3, an embodiment of selected display pages of one implementation of the GUI 112 is further described below, and is schematically illustrated in FIGS. 4-10, in order to illustrate how the expert system driven GUI 112 provides an efficient interface for the surgical team 110. The sequence of display pages followed during a surgical procedure may vary, depending on the selections by the surgical team.

Each of the display pages in accordance with the illustrated embodiment is visually divided into a top part, a middle area, and a bottom part. The bottom part of the GUI 112 includes an action bar 300 that displays an affirmation action button 302 and a negation action button 304 (although any widget of equivalent effect could be used). A uniform view of the options presented to the surgical team by the expert system is maintained to simplify the interface with the surgical team. Rather than presenting a number of options to the surgical team 110, the action bar 300 provides for the affirmation, or negation of a current option presented to the surgical team, and a main menu button 306 for accessing the main menu display page 204 (two instances of which are shown in FIGS. 4,6). As the foot-operated input device 122 provides activation of the affirmation and negation action buttons 302,304, hands-free access to the functionality of the CAS application is facilitated. As will be recognized by those skilled in the art, hands-free operation is important in many surgical procedures.

The action buttons (i.e. buttons on the action bar 300) are represented in a state that provides visual information about the accessibility of the button. The effect of triggering the action button, and a state of accessibility of the action button, is generally dependent on selections made in the top part and middle area of the display page that presents the action button. As described above, if an action button is not accessible, it is grayed-out; if the action button has been activated, it is illustrated as a depressed button; if the action button is a suggested action by the expert system, the action button is intermittently illuminated (i.e. “flashing”); otherwise, a normal view of the action button is presented, indicating that the action button can be selected even though it is not suggested. If the action button is deactivated, a mouse cursor cannot be used to select the action button, and a corresponding key on the keypad 114 is ineffectual. An audio tone may be associated with an attempt to select a grayed-out button.

With reference to particular display pages (illustrated in FIGS. 4-10), these states are exhibited. When the main menu 204 is displayed, the main menu button 206 is not accessible, and is accordingly grayed-out (FIGS. 4,6). Further, in the validate image display page 236 shown in FIG. 8 the main menu button 206 is grayed-out to ensure that a currently selected fluoroscopic image is either validated or rejected, to prevent the fluoroscopic image from remaining in an ambiguous state. The main menu button 206 is otherwise available at each of the display pages illustrated.

On both illustrated instances of the main menu 204, the expert system suggests the selections highlighted (in the top parts and middle areas of the display pages) and accordingly the affirmation action buttons 302 on these two pages are flashing. The negation action buttons 304 are also grayed-out as there is no negative action associated with the selection identified by the top parts and middle areas of the display pages.

In the calibrate U-handle display page 220 shown in FIG. 5, both the affirmation and negation action buttons 302,304 are grayed-out as the actions taken by the surgical team are on the instruments themselves. As the last step in the instrument calibration procedure, the U-handle is placed within a field of view of the tracking system, which automatically calibrates the surgical instrument. During this last step, a completion bar is overlaid on the calibrate U-handle display page 220 and the negation action button 304 is displayed in the normal (ready for activation) state, and is annotated with text indicating that the calibration process will be canceled if the negation action button 304 is selected. Similarly the affirmation and negation action buttons 302,304 are grayed-out in FIG. 10, because the function of the illustrated install implant display page 258, is to permit a visual of the insertion, and to permit the selection of the implant size and type; none of which requires the action buttons.

In FIGS. 7 and 8 both the affirmation and negation action buttons 302,304 are displayed in the ready state. Further in FIG. 9 the negation action button 304 is in the ready state, whereas the affirmation action button 302 is not available. At this juncture, activation of the negation action button 304 deletes a selected fluoroscopic image.

It will further be noted with reference to FIGS. 4-10, that the affirmation action button 304 and negation action button 302 are annotated with text that indicates a response to a corresponding presented option whenever the action button is not in a deactivated state. Accordingly, in FIGS. 4,6 the affirmative action button 302 annotated with “accept” is associated with initiating a selected procedure step (specifically the calibration of the universal handle, and the acquisition of a fluoroscopic image, respectively). In FIG. 7 the affirmation action button 302 is annotated with the text “Calibrate”, while the negation action button 304 is annotated with “Delete” a fluoroscopic image. The “Delete” annotation on the negation action button 304 is also present on the prepare implant site display page 254 and effects the same action. The affirmative action button 302 presented on the validate image display page 236 shown in FIG. 8 is annotated with the text “Accept”, indicating satisfactory agreement between the virtual instrument with respect to the fluoroscopic image, and the actual instrument with respect to the part of the person. The negation action button 304 on the validate image display page 236 is annotated with “Reject” and is also used to delete a selected fluoroscopic image on the basis that it is not in acceptable alignment with a calibrated instrument.

Main Menu Display Page Format

FIGS. 4,6 schematically illustrate two instances of an exemplary main menu display page 204 displayed by GUI 112 on the display monitor 106. The instance shown in FIG. 4 is consistent with an initial presentation of the main menu 204, displayed once the preliminary information about the surgery has been entered. In the instance shown in FIG. 4, the main menu 204 is presented once the expert system asserts that instrument calibration is complete.

In accordance with the illustrated embodiment, the top part of the main menu 204 displays a menu bar 310. The menu bar 310 includes four menu bar icons 316-322 each representing a respective one of the categories of functionality shown in FIG. 3; namely: the instrument calibration selection 206 represented by an instrument calibration icon 316 that resembles the three dimensional instrument locator 119; the patient imaging selection 208 represented by a patient imaging icon 318 resembling the fluoroscope 117; the implant preparation and installation selection 210 represented by an implant icon 320 that resembles a vertebra containing an implant; and the setup selection 212 represented by a setup icon 322 that, in other embodiments is associated with the preliminary display pages 202. It will be noted that the three icons relevant to performing the surgical procedure (icons 316-320) are grouped together, whereas the setup icon 322 is visually separated. The menu bar 310 further includes a right and a left button 324a,324b (which could be replaced by any equivalent widget) that is associated with a respective key on the keypad 114 and indicates the keys that can be used to change the selected menu bar icon (or other vertically offset selection set in procedural display pages). The right and left buttons 324a,324b are preferably also selectable by the mouse, if such an input device is available.

A highlighted border around a selected menu bar icon (as shown in FIG. 4, the instrument calibration icon 316) indicates which of the categories of functionality is currently active. Grayed-out menu bar icons (like other widgets) indicate that the menu bar icon (such as implant icon 320, as schematically shown on both FIGS. 4,6) are not available for selection, in a manner similar to the action buttons. In accordance with the illustrated embodiment of the invention, when the main menu 204 is displayed, each time the right or left button 324a,324b is selected a menu bar icon that is right-adjacent or left-adjacent to a currently selected menu bar icon becomes the selected menu bar icon, wherein right and left adjacency are determined by the visual order with the further specification that the setup icon 322 is left-adjacent to the instrument calibration icon 316, and reciprocally, the instrument calibration icon 316 is right-adjacent to the setup icon 322. In accordance with some embodiments of the invention, the grayed-out icons are by-passed. For example, when an icon right-adjacent to the selected icon is not available, and therefore grayed-out, activation of the right button 324a does not bring about the selection of the grayed-out icon, but rather selects the menu bar icon that is right-adjacent to the grayed-out button, if that button is available. Alternatively, grayed-out menu bar icons can be selected, however no options are available for selection in a middle area of the main menu 204, and both affirmation and negation action buttons 302,304 are grayed-out and not available, when a grayed-out menu bar icon is selected.

The middle area of the main menu 204 displays one or more selections associated with a currently selected menu bar icon. As the instrument calibration icon 316 is selected in the main menu 204 shown in FIG. 4, the middle area selections presented form a list of surgical instruments that may be used in the surgical operation identified during initial setup. As an organizational feature of the information displayed in the middle area, the selections are divided into tasks 334 and options 336. The task selections (i.e. selections presented within a task field, such as the calibrate U-handle selection 216) are deemed mandatory by the expert system, unlike optional selections (i.e. selections presented within an option field, such as the drill guide selection 218) are optional. It will be noted that the calibrate U-handle selection 216 is highlighted by a text-background color inversion, although any other highlighting scheme could be used, to indicate which of the selections qualifies the option presented to the surgical team. Accordingly, the action currently suggested by the expert system is the acceptance of the U-handle selection 216, which is presented when the instrument calibration icon 316 is active. The surgical team further has an option of choosing another selection from the task or option fields 334,336 using up or down buttons 342a,342b. Changing the highlighted selection in the middle area using the up and down buttons 342a,342b in relation to grayed-out selections is analogous to the operation of the right and left buttons 324a,324b. The up, down, right, and left buttons 342a,342b,324a,324b are embedded in lists, menus, etc. and are used for changing selections, menu options, etc. on different display pages of the GUI 112, permitting the efficient use of the keys on the keypad 114. As will be apparent to those skilled in the art, any number of layouts that provide the above-described functionality in a user friendly and accessible manner can also be used in embodiments of the invention.

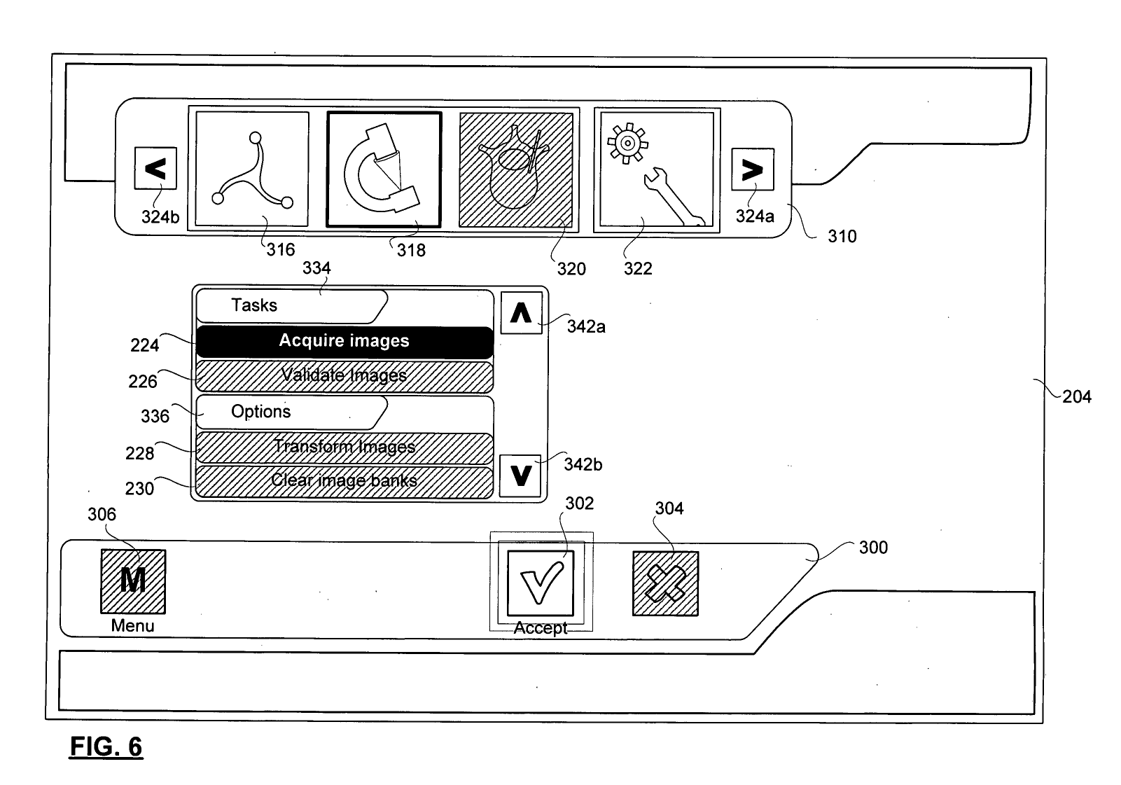

The middle area of the main menu 204 shown in FIG. 6 displays selections associated with the patient imaging icon 318 that is currently selected. Specifically the task field includes the acquire images setup selection 224, and the validated images selection 226, the former of which is highlighted. The option field includes the transform images selection 228, and the clear image bank selection 230. As only the acquire images selection 224 is in the ready state (and all others are grayed-out), the effect of the up and down buttons 342a,342b is null.

Procedural Display Page Format

Display pages 220, 232, 236, 254 and 258 shown in FIGS. 5,7,8,9, and 10, respectively, are termed “procedural” display pages as they relate to steps in the computer assisted surgical procedure. An orientation bar 350 is featured on the top part of the procedural display pages for providing information relating to the current step in the CAS 100. Specifically the orientation bar 350 includes a place for the menu bar icon that indicates the category of functionality accessed (shown in a top left-hand corner), and a brief text field 352 for identifying a current step in the surgical procedure, notifying the surgical team of available commands, etc. Naturally the instrument calibration icon 316 is presented on the calibrate U-handle display page 220; the patient imaging icon 318 is presented on the acquire fluoroscope image display page 232 and the validate image display page 236; and the implant icon 320 is presented on the orientation bar 350 of the prepare implant site display page 254, and the install implant display page 258.

The orientation bar 350 further displays a plurality of instrument icons 354. Specific instrument icons 354 are identified as follows: U-handle instrument icon 354a (shown on procedural display pages 220,236,254, 258); C-arm instrument icon 354b (shown on acquire fluoroscope image display page 232); clamp instrument icon 354c (shown in procedural display pages 232,236,254, 258); and drill instrument icon 354d (shown on the prepare implant site display page 254). The instrument icons 354 presented on a procedural display page indicate which of the calibrated instruments (or instruments in the process of being calibrated) are expected to be used in the current step of the surgical procedure. These indicated instruments may be presented in either a normal view, indicating that the instrument associated with the instrument icon 354 is within the field of view of the tracking system, or in a grayed-out view to indicate that the associated instrument is not within the field of view of the tracking system. For example, the drill instrument icon 354d on the prepare implant site display page 254 shown in FIG. 9 is grayed-out to indicate that the drill guide is not within the field of view of the tracking system.

The middle area of the procedural display pages displays one or more content fields related to a step in the surgical procedure that is underway. For example, calibrate U-handle display page 220 presents an illustrated guide 356 for securing the instrument locator 119 onto the universal handle, and placing the instrument locator 119 within the field of view of the tracking system.

The middle areas of each procedural display page that is displayed after a fluoroscopic image is acquired, preferably includes two image content fields: an anterior-posterior (A-P) image content field 358a, and a lateral (LAT) content field 358b, for displaying respective fluoroscopic images 359a,359b.

The fluoroscopic images 359a,359b are preferably X-ray images but alternative imaging (e.g. ultrasound) can be used for imaging the part of the patient. In accordance with the embodiment shown, a vertebral clamp is in a view of the fluoroscopic images 359a,359b and is rigidly secured to the spine of the patient. The clamp is used to automatically calibrate the fluoroscopic images 359a,359b during the calibration step.

In accordance with the illustrated embodiment, a number of AP and LAT fluoroscopic images may be acquired. Associated with each of the image content fields 358a,358b for which selection of the fluoroscopic image is possible (i.e. procedural display pages 232,236,254) is an image selection field 360, which, in the illustrated embodiment includes a numeral that identifies the fluoroscopic image, and the up and down buttons 342a,342b. The up button 342a is associated with a fluoroscopic image that has an incrementally higher identifier numeral, and conversely, the down button 342b is associated with an image having an incrementally lower identifier numeral. In alternative embodiments, other image identifiers, and widgets for selecting images by their identifiers, can be used to provide intuitive user interaction. If only one image is acquired for display in one image content field, as is the case of the A-P image content field 358a in the acquire fluoroscope image display page 232 shown in FIG. 7, the up and down buttons 342a,342b are grayed-out.

The image content fields 358a,358b of the validate image display page 236, the prepare implant site display page 254, and the install implant display page 258 are shown with overlaid virtual instruments 370. More specifically the awl tip of the U-handle is displayed over the fluoroscopic image 359b of the validate image display page 236, and over the fluoroscopic images 359a,359b in the prepare implant site display page 254. The virtual instrument associated with the U-handle, having an appropriate tip is shown with a virtual implant 372 (which is a pedicle screw) on the install implant display page 258. In the illustrated embodiment of the prepare implant site display page 254, an axis 374 extends a predetermined distance from the center of the virtual instrument used to prepare the implant site, the axis being concentric with a major axis of the instrument. This axis 374 is useful for ensuring that the bored hole is well chosen. In some embodiments, the virtual implants remain displayed on the fluoroscopic images 359a,359b even after installation.

The patient imaging icon 318 is also displayed in each image content field. If the patient imaging icon 318 of an image content field is grayed-out(e.g. the LAT image content field 358b in FIG. 7), no image is available for viewing in the image content field, and a background 362 and image selection field 360 are grayed-out as well. If the patient imaging icon 318 contains an ellipsis, the image is in a process of being acquired (for example, see the A-P image content field 358a shown in FIG. 7). If the image is not yet validated, it is marked with an X, as shown in the LAT image content field 358b of FIG. 8, whereas the successfully validated image content field is marked with a check, as shown in the A-P image content field 358b of the same figure.

The image content fields 358a,358b may further include backgrounds 362 that can be emphasized or deemphasized to indicate which of the two image content fields is currently selected. In procedural display pages that permit the selection of fluoroscopic images, the surgical team is enabled to change the selection of the image content field using one of the right and left buttons 324a,324b. If the A-P image content field is currently selected (as in FIG. 7) the right button 324a is included in the A-P image content field, for alternating selection to the LAT image content field. Symmetrically, a LAT image content field that is currently selected (as in FIGS. 8,9) includes the left button 324b, for selection the A-P image content field. As the LAT image content field 358b shown in FIG. 7 is associated with an empty image bank, the right button 324a is grayed-out. Naturally the image selection field 360 of only the selected image content field is emphasized and includes the up and down buttons 342a,342b. It will be noted that the installation of the implant after the site has been prepared generally requires the same view of the implant site to be rendered because that image is most precisely calibrated with the drilled hole. Accordingly no option for changing displayed images is presented to the surgical team (i.e. no image selection field 360), during the implant installation procedure step, and backgrounds 362 of both image content fields are highlighted.

Other fields as required are defined for respective purposes related to respective procedure steps. For example, an implant size field 364 that includes widgets for changing a selected implant size (or alternatively size and type) is shown in FIG. 10. As is preferable, the implant size field 364 includes right and left buttons 324a,324b. In alternate embodiments of the invention, the middle area of the install implant display page 258 includes a virtual path overlaid on each of the fluoroscopic images 359a,359b that are concentric with the implant sites, the path being computed with respect to the axis of the implant, and an implant size. As the implant (e.g., a pedicle screw) is installed, the virtual path of the implant is displayed in a contrasting color over the prepared implant site 510, for example. Concurrently, a depth of the pedicle hole may be shown on a depth gauge as the insertion progresses. This permits the surgeon to monitor an axis of orientation and a depth of insertion of the implant.

Another example of a field used for a specific step in the surgical procedure is associated with the validate image display page 236. An illustrated description field 366 overlays the unselected image content field 358a in order to identify anatomical reference points used to verify the alignment of the calibrated instrument with the calibrated fluoroscopic image (displayed in image content field 358b). More specifically, the illustrated description field 358a displays arrows indicating points suggested by the expert system to be used to validate the fluoroscopic image. The surgical team places a calibrated instrument 118 on corresponding anatomical features of the patient. As the surgical team places the calibrated instrument (the U-handle with the awl tip, for example), the CAS computes a position of the awl tip with respect to the image to be validated (fluoroscopic image 359b) and the GUI 112 displays a virtual image of the instrument on the fluoroscopic image 359b. The actual location of the calibrated instrument is then compared with the virtual representation of the instrument on the fluoroscopic image 359b. If the actual location of the calibrated instrument 118 at the plurality of points on the part of the patient is indistinguishable from the position of the virtual representation of the instrument on the image to be validated, the image is validated and can be used for surgical purposes. The surgical team 110 accepts the validated image by pressing the affirmation action button 302. Alternatively, an audio tone may be sounded to indicate that the validated image has been saved.

The invention therefore provides an expert system driven graphical user interface that facilitates surgical procedures by guiding a surgical team through a surgical procedure, while providing critical information respecting the calibrations of the system, preparation of the implant site, and placement of implants.

The embodiment(s) of the invention described above is(are) intended to be exemplary only. The scope of the invention is therefore intended to be limited solely by the scope of the appended claims.

I/we claim:

1. A graphical user interface (GUI) for guiding a surgical team through a surgical procedure, the GUI comprising:
a series of display pages for providing information related to respective steps required to perform the surgical procedure, and for displaying to the surgical team representations of selected surgical instruments in a field of view of a tracking system overlaid on a fluoroscopic image of a subject of the procedure; and
action widgets for permitting the surgical team to advance through the series of display pages as each of the respective steps is successfully completed.

2. A GUI as claimed in claim 1 wherein the action widgets comprise at least one action widget for responding to a presented option on each of the series of display pages, the presented option being presented by an expert system associated with the surgical procedure, in response to actions and selections by the surgical team.

3. A GUI as claimed in claim 2 wherein the action widgets include an affirmation and a negation action button, each of the buttons being presented in a same region of corresponding display pages, and wherein the action buttons are represented in a state that indicates one of: activation, available for activation, suggested activation, and unavailable for activation.

4. A GUI as claimed in claim 3 wherein each of the action buttons in a state other than unavailable for activation is annotated by text associated with the presented option, and the action widgets further include a main menu button for accessing a main menu page.

5. A GUI as claimed in claim 3 wherein the series of display pages are organized in categories of functionality related to instrument calibration, patient imaging, implant site preparation, and implant installation.

6. A GUI as claimed in claim 5 wherein the display pages in the instrument calibration category comprise selections and content fields for permitting the surgical team to select surgical instruments, and calibrate the selected surgical instruments used to perform the surgical procedure.

7. A GUI as claimed in claim 6 wherein each of the display pages in any of: the instrument calibration, implant site preparation, and implant installation categories comprise a set of instrument icons representing respective calibrated instruments, the instrument icons being presented in a same part of the corresponding display pages, and wherein a state of each of the instrument icons indicates one of the following: the instrument is not used for a current procedure step, the instrument is outside the field of view, and the instrument is within the field of view.

8. A GUI as claimed in claim 7 wherein the display pages in the patient imaging category comprise selections and content fields for enabling the surgical team to:
control a fluoroscope to acquire images of a part of the patient; and
validate the acquired images to ensure alignment using the calibrated instruments.

9. A GUI as claimed in claim 8 wherein the display pages in the patient imaging and implant site preparation categories further comprise:
at least two image content fields associated with respective incidences of the part of the patient;
an image identifier in each image content field; and
an image selection widget permitting the surgical team to select an image to be displayed in a selected image content field from among one or more banks of images.

10. A GUI as claimed in claim 9 wherein the at least two image content fields are also displayed in display pages in the implant installation category, and each image content field further comprises a patient imaging icon that is represented in a state indicating one of the following: that no image is available for an associated view; that an image is being acquired; that an image is acquired but has not been validated; and that an image is validated.

11. A method for guiding a surgical team in performing a surgical procedure using a graphical user interface (GUI), the method comprising:
providing the surgical team with information related to respective procedure steps required to perform the surgical procedure using the GUI;
displaying to the surgical team representations of selected surgical instruments used during the surgical procedure, in alignment with a fluoroscopic image of a part of a patient that is subject to the surgical procedure; and
advancing through a series of display pages designed for the surgical procedure, as each of the respective procedure steps is successfully completed.

12. A method as claimed in claim 11 wherein advancing through a series of display pages comprises activating an action widget on each of the display pages for responding to a presented option, the presented option being presented by an expert system associated with the surgical procedure, in response to actions and selections by the surgical team via the GUI.

13. A method as claimed in claim 12 wherein the selecting an action widget comprises activating one of an affirmation and a negation action button, each of the buttons being presented in a same region of each of the display pages, and wherein a state of each of the action buttons indicates one of: activation, available for activation, suggested for activation, and not available for activation.

14. A method as claimed in claim 13 wherein activating one of an affirmation and a negation action button comprises selecting an action button that is in a state other than not available for activation, the action button being annotated with text associated with the presented option.

15. A method as claimed in claim 14 wherein providing the surgical team with information related to procedure steps involves providing content fields in display pages related to one of instrument calibration; patient imaging; and implant preparation and installation.

16. The method as claimed in claim 15 wherein providing content fields in display pages related to instrument calibration prompts the surgical team to apply a sequence of procedures to calibrate a selected instrument, and wherein providing content fields in display pages related to patient imaging, implant site preparation, and implant installation include providing instrument icons located in a same position on the display pages and being presented in a state to indicate one of the following: the calibrated instrument is not required in a current procedure step; the calibrated instrument is within the field of view of the tracking system; and the calibrated instrument is outside of the field of view.

17. A method as claimed in claim 16 wherein providing content fields in display pages related to patient imaging comprises providing display pages that prompt the surgical team to acquire and validate fluoroscopic images at one or more substantially different incidences of the part of the patient, and wherein providing content fields in display pages related to patient imaging and implant site preparation, include presenting two image content fields for displaying corresponding fluoroscopic images of the part of the patient, each of the image content fields further comprising an image identifier of a currently displayed image, and an image identifier widget permitting the surgical team to select another image identifier to be displayed in the image content field.

18. A system for performing a computer-assisted surgical procedure, the system comprising:
a computer including a video display supporting a graphical user interface (GUI) for guiding a surgical team in the performance of the surgical procedure, wherein the GUI includes a series of display pages for providing information related to respective steps required to perform the surgical procedure, and for displaying virtual images of selected surgical instruments within a field of view of a tracking system in relative alignment with a fluoroscopic image of a part of a patient subject to the surgical procedure;
the tracking system for tracking a location of the selected surgical instruments with respect to the part of the patient to permit overlaying of the virtual images of the selected surgical instruments on the oriented image of the part of the patient;
an imaging system for acquiring images of the part of the patient used to create the oriented image; and
means for permitting the surgical team to advance through the series of display pages as each of the respective steps is completed.

19. A system as claimed in claim 18 wherein the imaging system comprises one of a fluoroscope, MRI, CT-Scan, PET, ultrasound, and echography machine connected to the computer.

20. A system as claimed in claim 18 wherein the tracking system comprises:
a binocular visual system connected to the computer; and
a light-reflective reference tool connected to each of the selected surgical instruments that identifies an orientation and position of the surgical instrument.

21. A system as claimed in claim 18 further comprising a data network for connecting a remote data source to the computer.

22. A system as claimed in claim 18 wherein the means for permitting the surgical team to advance through the series of display pages comprises a manual input device connected to the computer.

23. A system as claimed in claim 18 wherein the means for permitting the surgical team to advance through the series of display pages comprises a foot-operated input device connected to the computer.

24. A system as claimed in claim 23 wherein the foot-operated input device comprises two pedals that are associated with affirmation and negation actions, respectively, the affirmation and negation actions constituting respective responses to an option presented by the display pages of the GUI, the option being presented by an expert system associated with the surgical procedure, in response to actions and selections by the surgical team.

25. A system as claimed in claim 24 wherein the affirmation and negation actions are associated with two corresponding keys of a manual input device, and two action widgets that are presented in a same position in each of the display pages.

26. A system as claimed in claim 25 wherein each of the two action widgets is a corresponding action button that is annotated with text associated with the presented option.
